(19) United States
(12) Patent Application Publication
(10) Pub. No.: US 2009/0234356 A1
(43) Pub. Date: Sep. 17, 2009

(54) METHOD AND APPARATUS FOR FRACTURE FIXATION

(76) Inventor: Barry T. Bickley, North Andover, MA (US)

Correspondence Address: Mark J. Pandiscio;Pandiscio & Pandiscio, P.C., 470 Totten Pond Road, Waltham, MA, 02451, US

(21) Appl. No.: 12/264,039

(22) Filed: Nov. 3, 2008

Related U.S. Application Data
(60) Provisional application No. 61/001,599, filed on Nov. 2, 2007.

(51) Int. Cl. A61B 17/00

(52) U.S. Cl. 606/59

(57) ABSTRACT

A method for securing a first bone segment to a second bone segment, the method comprising the steps of:

positioning the first bone segment against the second bone segment, forming a first bore through the first bone segment and into the second bone segment, and forming a second bore into the second bone segment so that the second bore intersects the first bore;

positioning a locking member within the second bore so that an opening in the locking member is aligned with the first bore; and

positioning an interlocking screw within the first bore so that the interlocking screw passes through the opening in the locking member and makes a locking engagement therewith, whereby to lock the interlocking screw with the locking member and thereby secure the first bone segment to the second bone segment.

A system for securing a first bone segment to a second bone segment, wherein a first bore is formed through the first bone segment and into the second bone segment, and a second bore is formed into the second bone segment so that the second bore intersects the first bore of the first bone segment, the system comprising:

a locking member for positioning within the second bore so that an opening in the locking member is aligned with the first bore; and

an interlocking screw for positioning within the first bore so that the interlocking screw may be passed through the opening in the locking member and make a locking engagement therewith, whereby to lock the interlocking screw with the locking member and thereby secure the first bone segment to the second bone segment.

### REFERENCE TO PENDING PRIOR PATENT APPLICATION

This patent application claims benefit of pending prior U.S. Provisional Patent Application Ser. No. 61/001,599, filed Nov. 2, 2007 by Barry T. Bickley for METHOD AND APPARATUS FOR FRACTURE FIXATION (Attorney's Docket No. SIM-4 PROV), which patent application is hereby incorporated herein by reference.

### FIELD OF THE INVENTION

This invention relates to surgical methods and apparatus in general, and more particularly to methods and apparatus for fracture fixation.

### BACKGROUND OF THE INVENTION

Bone fractures are a relatively common physical malady. Bone fractures are typically the result of a traumatic injury, e.g., a fall, an automobile accident, etc. Bone fractures can also be brought on by certain medical conditions which significantly weaken bones, e.g, osteoporosis, cancer, etc.

When a bone is fractured, it is common to temporarily stabilize the bone with a stabilization apparatus while healing occurs. This process is commonly referred to as fracture fixation. The stabilization apparatus may comprise external devices (e.g., casts, support frames, etc.) and/or internal devices (e.g., plates and screws, intramedullary rods and screws, cerclage wires, etc.).

One common type of fracture occurs at the base of the tibia. More particularly, and looking now at FIG. 1, there is shown a tibia 5. Tibia 5 is characterized by, among other things, a distal end 10 having a lateral protusion (medically referred to as “the lateral malleolus”) 15. Distal end 10 of tibia 5 terminates in a bearing surface 20 which slidably engages the talus bone (not shown) so as to form the ankle joint. In many cases, the tibia may be fractured at the point where lateral malleolus 15 meets the remainder of the tibia, e.g., such as at the fracture line 25. In this case, it is frequently necessary to stabilize lateral malleolus 15 at fracture line 25 while healing occurs.

In this respect it should be appreciated that, while fracture fixation is commonly employed for many different types of bone fractures, it is particularly important in the case of a fracture of the lateral malleolus. This is because the ankle's bearing surface 20 extends along a portion of the lateral malleolus and, as a result, it is critical that the lateral malleolus be properly stabilized during healing if the patient is to fully recover from the fracture.

Currently, a fracture of the lateral malleolus is typically stabilized by passing one or more screws through the lateral malleolus and into the distal end of the tibia. See, for example, FIG. 2, where a screw 30 is shown extending through lateral malleolus 15 and into distal end 10 of tibia 5. However, this stabilization technique is hampered by the fact that the integrity of the repair is entirely dependent upon (i) maintaining proper alignment of the lateral malleolus while installing the screw, through the lateral malleolus and into the tibia, and (ii) the quality of the bone at the distal end of the tibia. In point of fact, many surgeons have difficulty maintaining proper bone alignment while setting the screw. Perhaps even more importantly, many patients (particularly older patients) have relatively poor bone quality at the distal end of the tibia, and the threads of the screw cannot gain sufficient purchase with the surrounding tibia to effect a reliable fracture fixation.

Thus there is a need for a new and improved approach for stabilizing the lateral malleolus while healing occurs.

There is also a need for a new and improved approach for stabilizing other fractures while healing occurs.

### SUMMARY OF THE INVENTION

These and other objects of the present invention are addressed by the provision and use of a new and improved fracture fixation system.

In one form of the invention, there is provided a method for securing a first bone segment to a second bone segment, the method comprising the steps of:

positioning the first bone segment against the second bone segment, forming a first bore through the first bone segment and into the second bone segment, and forming a second bore into the second bone segment so that the second bore intersects the first bore;

positioning a locking member within the second bore so that an opening in the locking member is aligned with the first bore; and

positioning an interlocking screw within the first bore so that the interlocking screw passes through the opening in the locking member and makes a locking engagement therewith, whereby to lock the interlocking screw with the locking member and thereby secure the first bone segment to the second bone segment.

In another form of the invention, there is provided a system for securing a first bone segment to a second bone segment, wherein a first bore is formed through the first bone segment and into the second bone segment, and a second bore is formed into the second bone segment so that the second bore intersects the first bore of the first bone segment, the system comprising:

a locking member for positioning within the second bore so that an opening in the locking member is aligned with the first bore; and

an interlocking screw for positioning within the first bore so that the interlocking screw may be passed through the opening in the locking member and make a locking engagement therewith, whereby to lock the interlocking screw with the locking member and thereby secure the first bone segment to the second bone segment.

### BRIEF DESCRIPTION OF THE DRAWINGS

These and other objects and features of the present invention will be more fully disclosed in, or rendered obvious by, the following detailed description of the preferred embodiments of the invention, which is to be considered together with the accompanying drawings wherein like numbers refer to like parts and further wherein:

FIG. 1 is a schematic view of a tibia with a fractured lateral malleolus;

FIG. 2 is a schematic view of an X-ray showing a prior art repair for a fractured lateral malleolus;

FIGS. 3-7 are schematic views showing a fracture fixation system formed in accordance with the present invention, including its various components and their features, and their manner of assembly;

FIGS. 8-29 are schematic views showing the repair for a fractured lateral malleolus using the present invention;

FIG. 30 is a schematic view of showing the repair of an olecranon fracture of the ulna; and

FIGS. 31-37 show alternative constructions for the present invention.

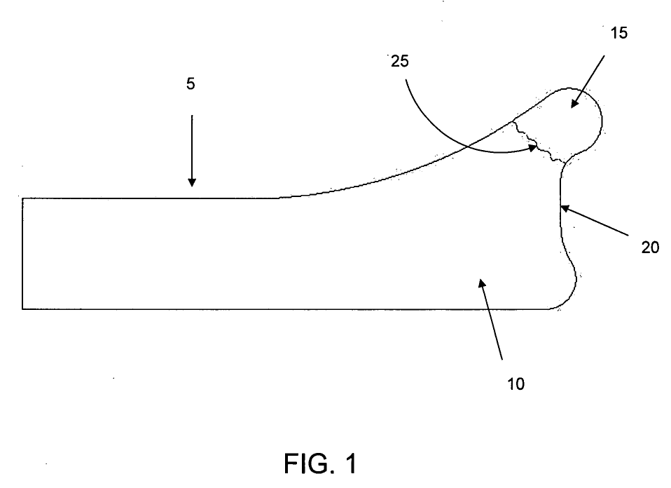

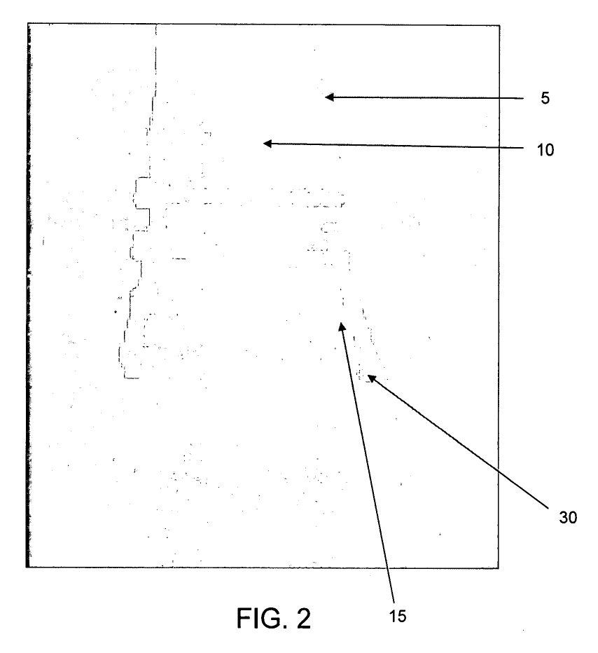

### DETAILED DESCRIPTION OF THE PREFERRED EMBODIMENTS OF THE INVENTION

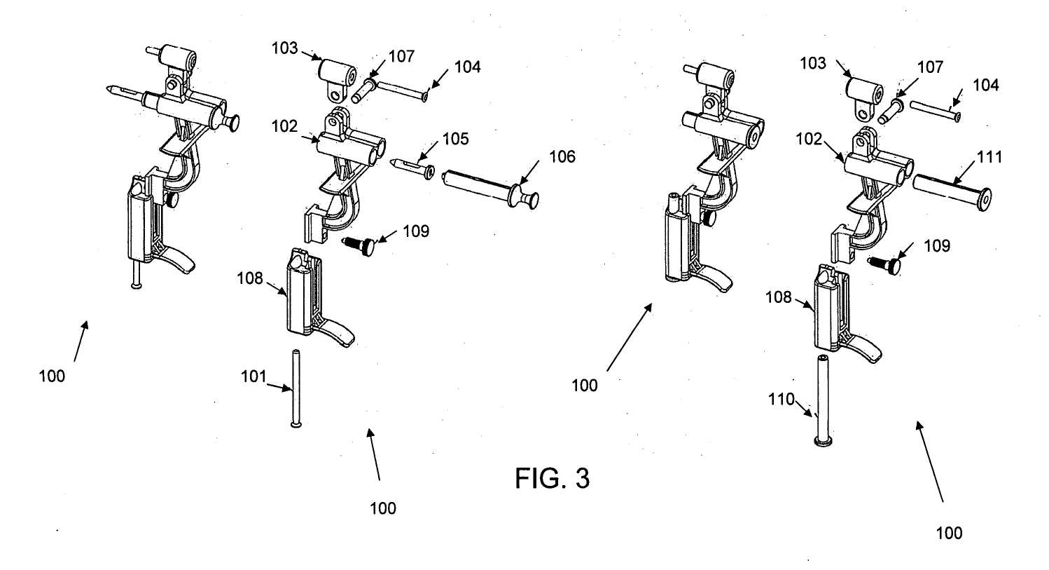

Looking now at FIG. 3, the present invention comprises the provision and use of a new facture fixation system 100. Fracture fixation system 100 generally comprises an interlocking screw 101, an arm 102, a pivot block 103, an anchoring screw 104, a locking member 105, an upper tube pusher 106, a clevis pin 107, lower ratchet tubes 108, a spring plunger 109, a lower tube cannula 110 and an upper tube cannula 111. As seen in FIG. 3, and as will hereinafter be described, pivot block 103 is rotatably pinned to arm 102, and lower ratchet tubes 108 are slidably connected to arm 102.

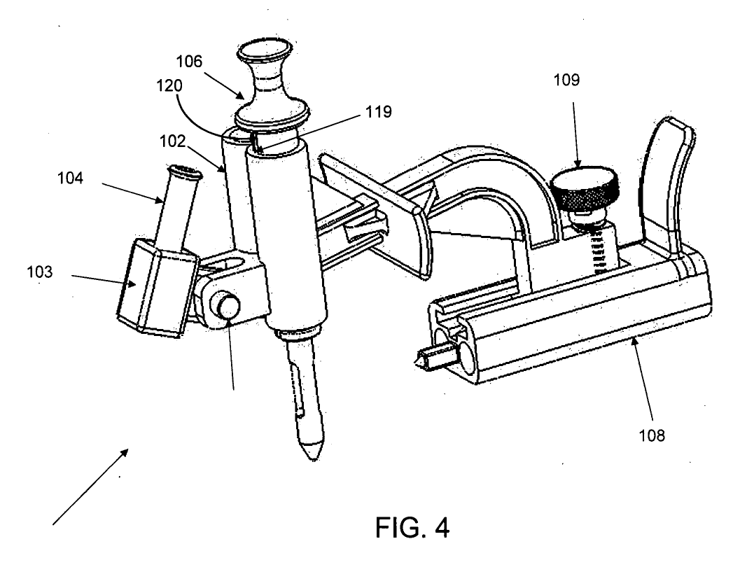

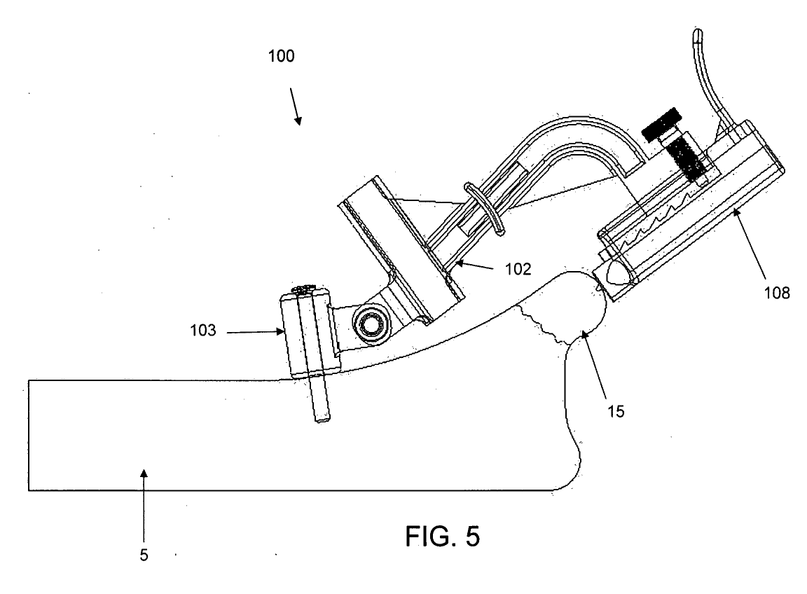

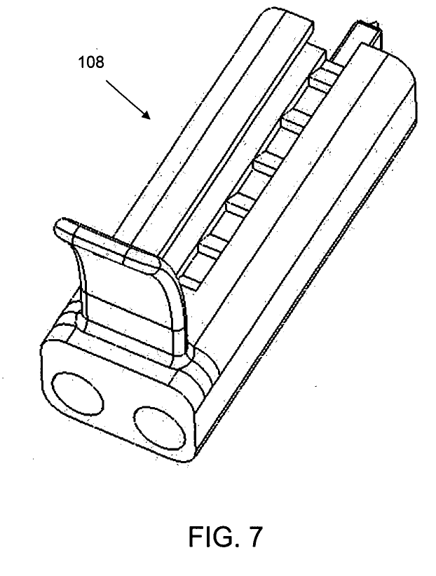

FIGS. 4-7 show further details with respect to fracture fixation system 100, its various components and their features, and their manner of assembly, all of which will be discussed hereinafter.

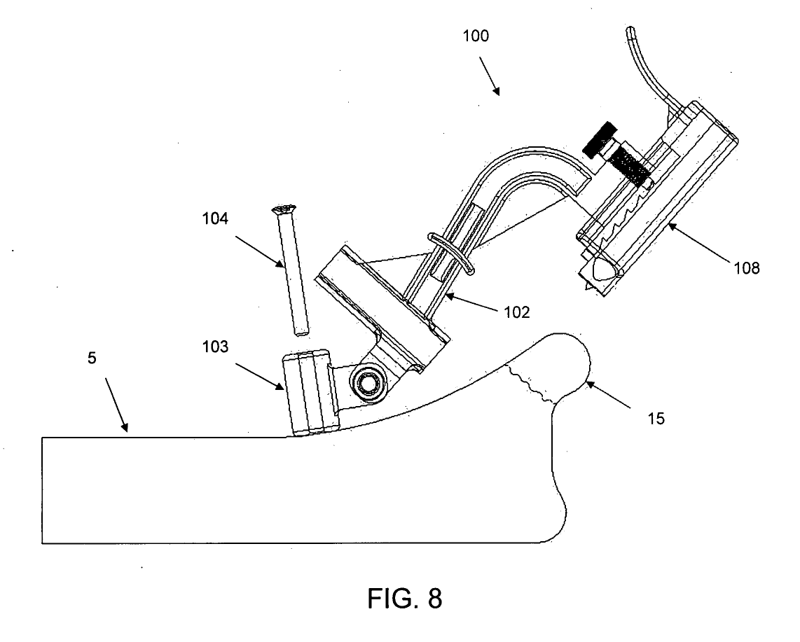

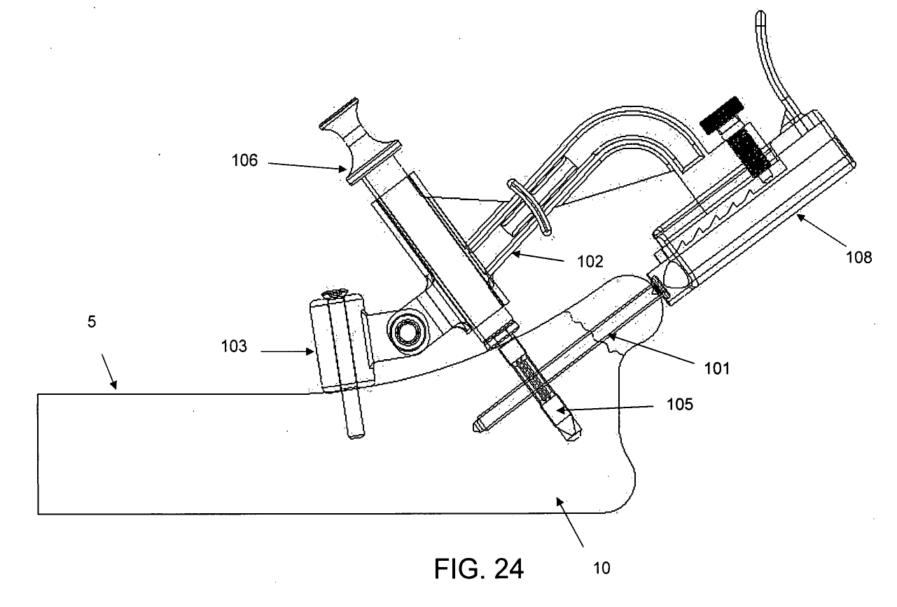

FIGS. 8-29 illustrate how fracture fixation system 100 may be utilized to stabilize a fracture of the lateral malleolus while healing occurs.

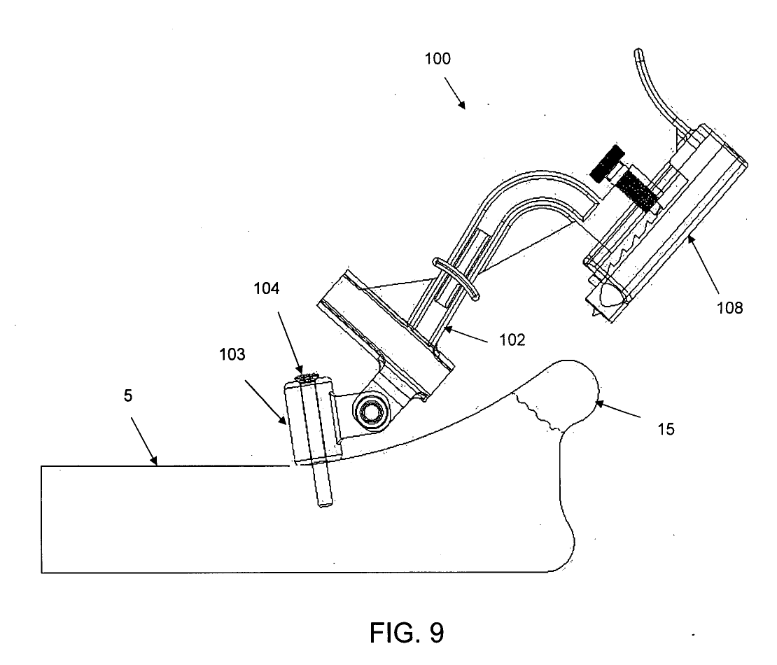

More particularly, as shown in FIGS. 8 and 9, fracture fixation system 100 is secured to tibia 5 by passing the anchoring screw 104 through pivot block 103 and into tibia 5.

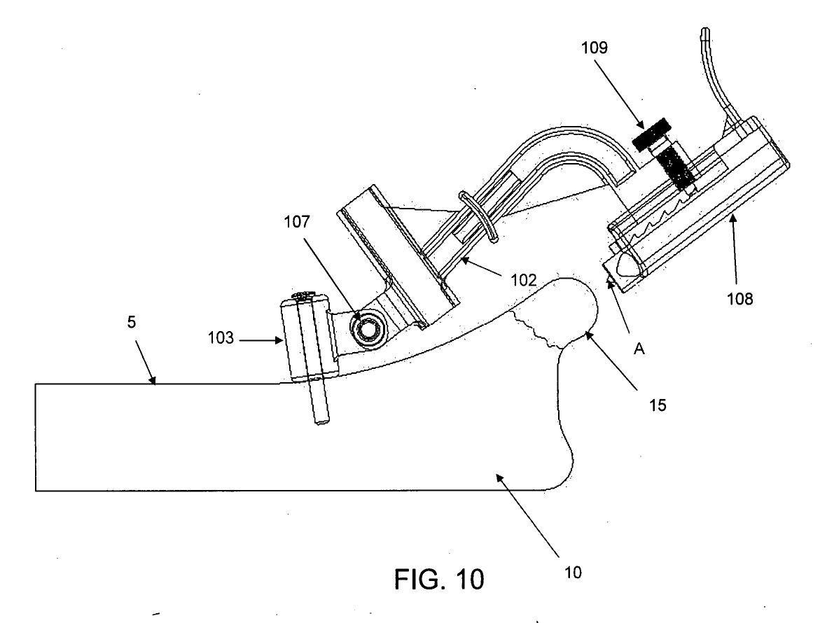

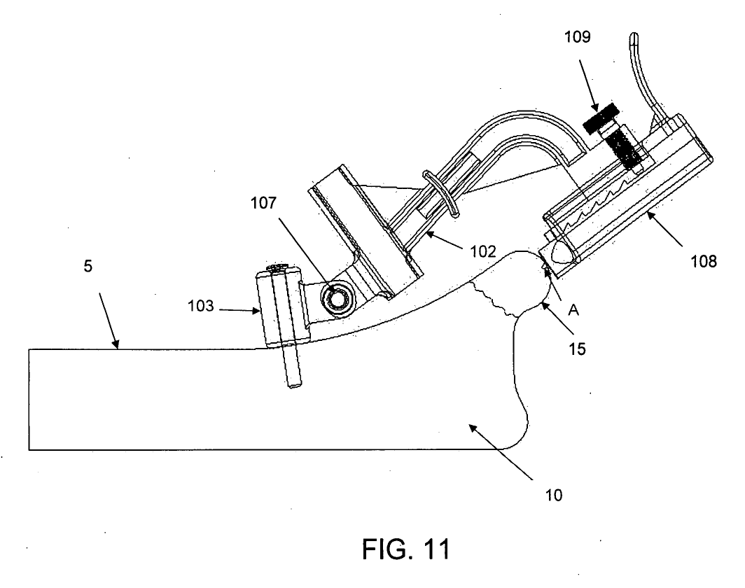

Then, as shown in FIGS. 10 and 11, lower ratchet tubes 108 are first brought into alignment with lateral malleolus 15 by rotating the system about clevis pin 107 (FIG. 10), and then brought into engagement with lateral malleolus 15 by longitudinally advancing lower ratchet tubes 108 against the force of spring plunger 109. At this point, the distal tip A of the lower ratchet tubes will compressibly hold the lateral malleolus in position against the remainder of the tibia.

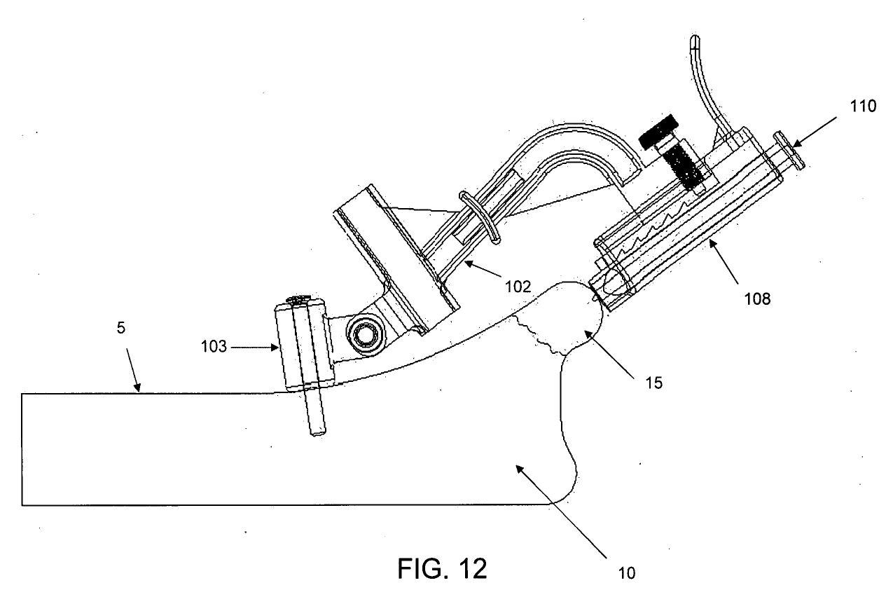

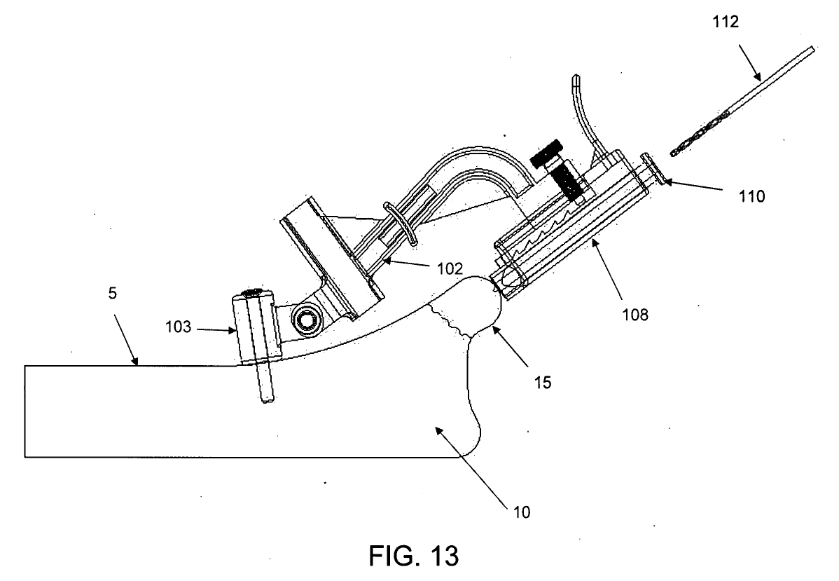

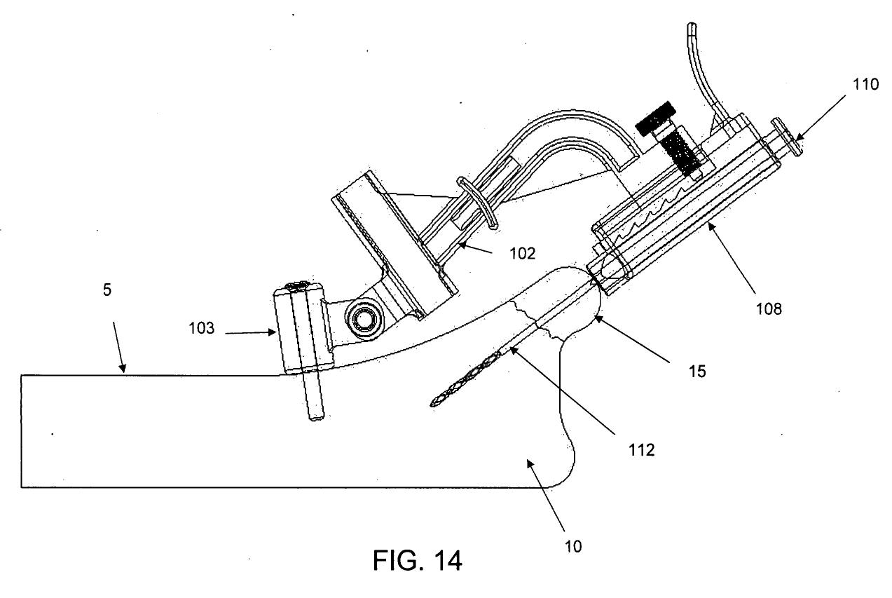

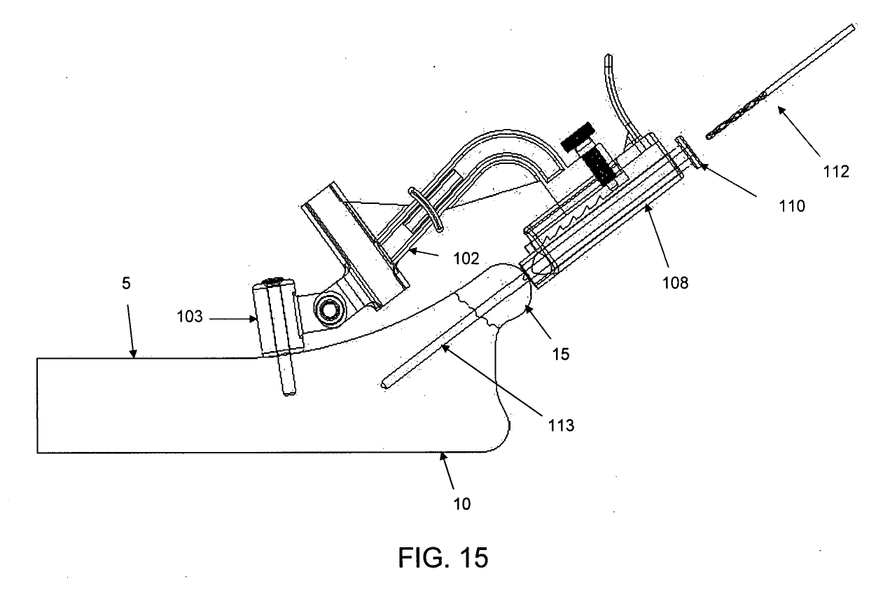

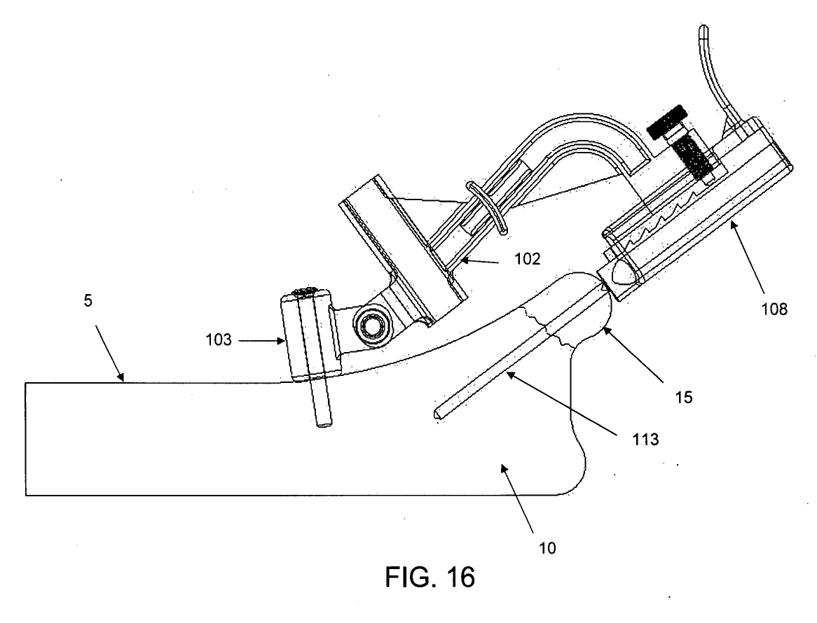

Looking next at FIGS. 12-16, lower tube cannula 110 is next inserted into one of the bores of lower ratchet tubes 108 (FIG. 12), a drill 112 is passed down lower tube cannula 110, through the lateral malleolus and into the distal end of the tibia (FIGS. 13 and 14), and then withdrawn (FIG. 15), and then lower tube cannula 110 is withdrawn from the bore of lower ratchet tubes 108 (FIG. 16), leaving a bore 113 extending through lateral malleolus 15 and into distal end 10 of tibia 5. Then this process is repeated for the other of the bores of lower ratchet tubes 108 (not shown). At this point, two holes will have been drilled through the lateral malleolus and into the distal end of the tibia. At the same time, the lateral malleolus will be held in position against the distal end of the tibia by virtue of the compressive engagement of lower ratchet tubes 108.

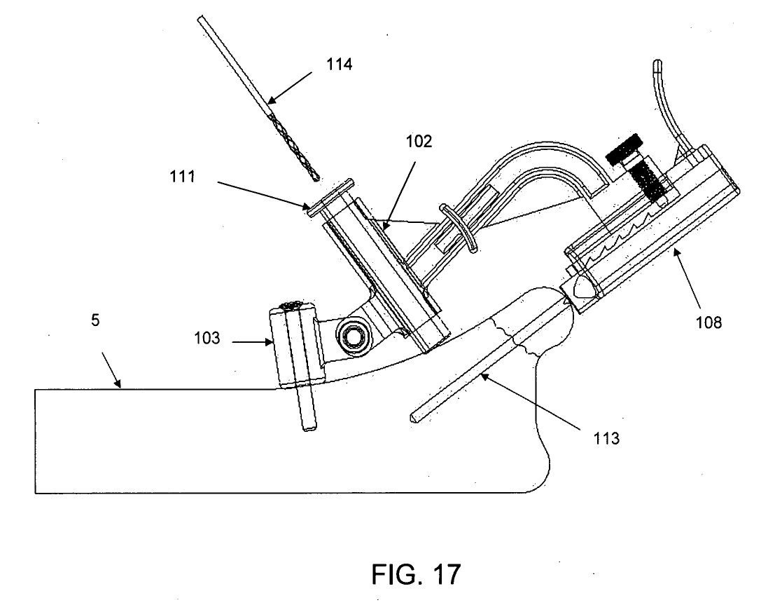

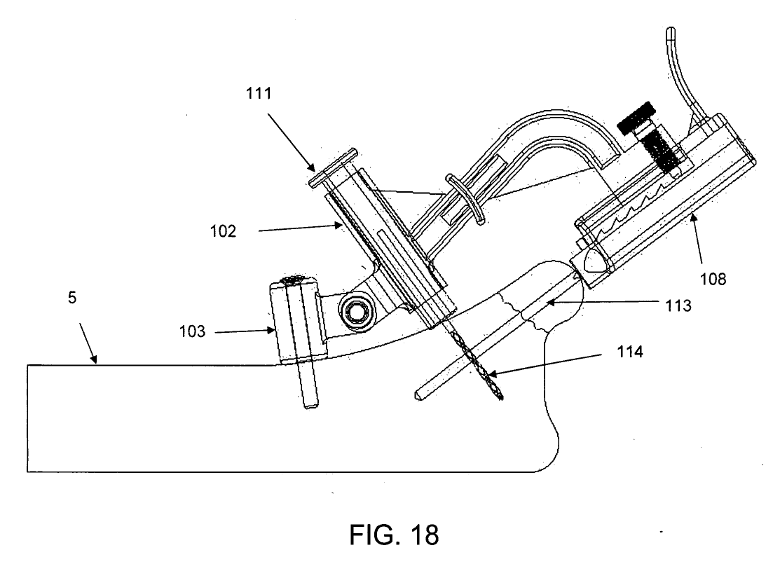

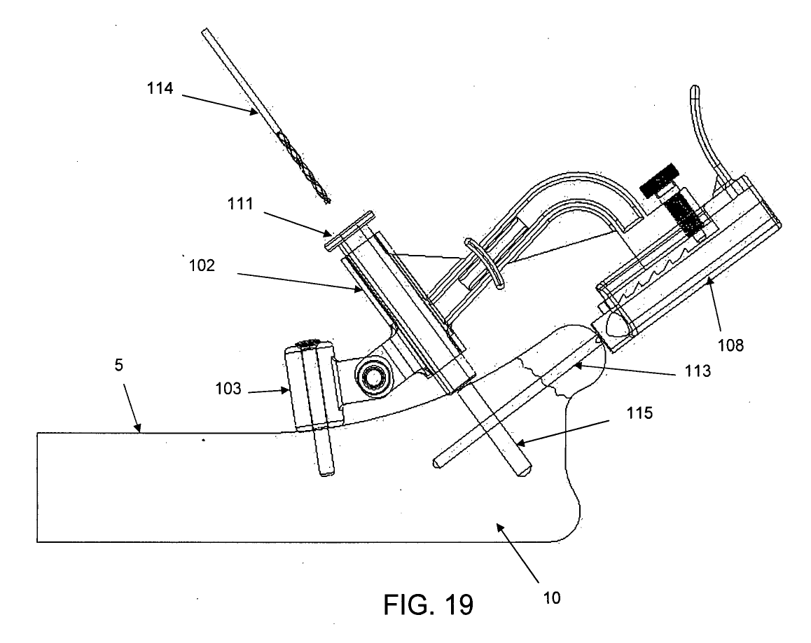

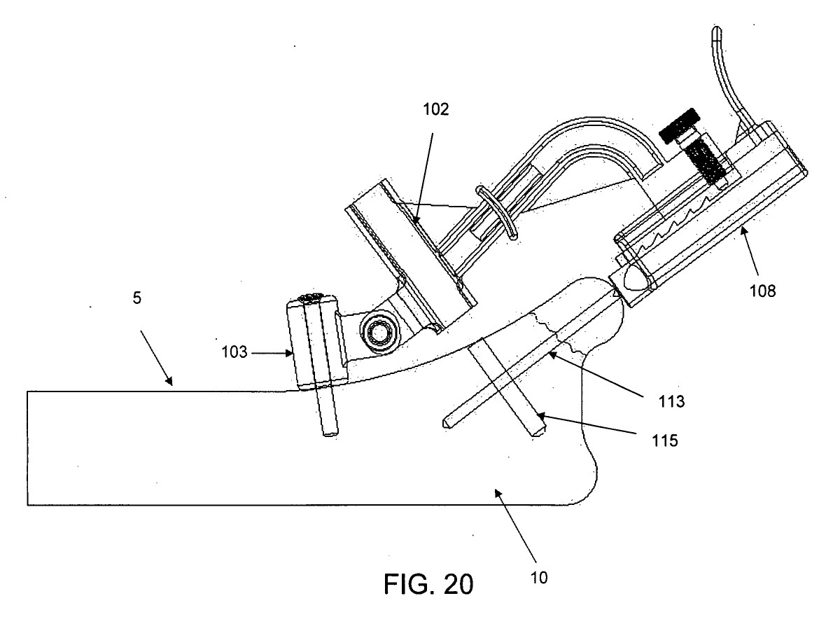

Looking now at FIGS. 17-20, upper tube cannula 111 is then inserted into one of the bores of arm 102 (FIG. 17), a drill 114 is passed down upper tube cannula 111 and into tibia 5 (FIGS. 17 and 18), and then drill 114 is withdrawn (FIG. 19), leaving a bore 115 extending through distal end 10 of tibia 5. Next, upper tube cannula 111 is withdrawn (FIG. 20). Then this process is repeated for the other of the bores of the upper ratchet tubes 111 (not shown). At this point, two holes will have been drilled into the tibia. Significantly, due to the construction of fracture fixation system 100, each of the holes drilled through upper tube cannula 111 will precisely intersect one of the holes drilled through lower tube cannula 110.

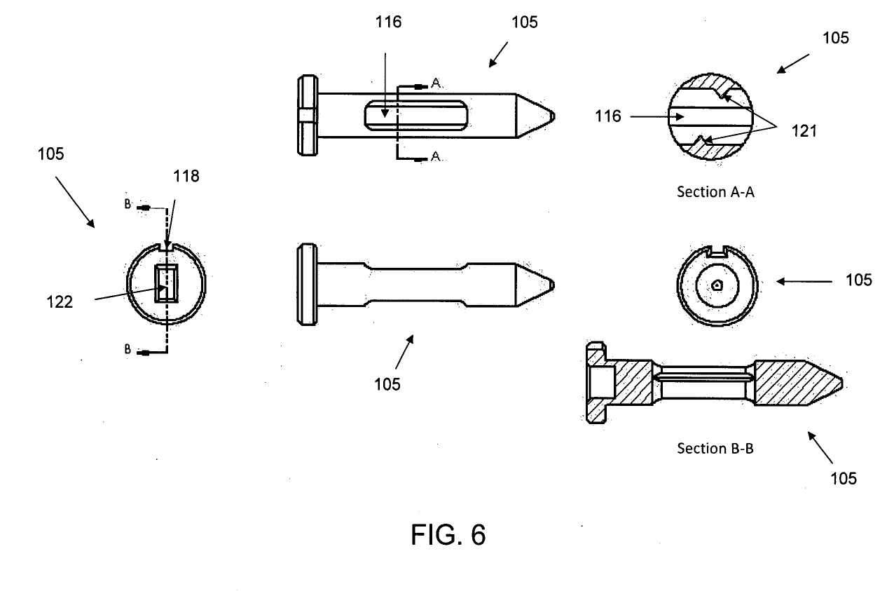

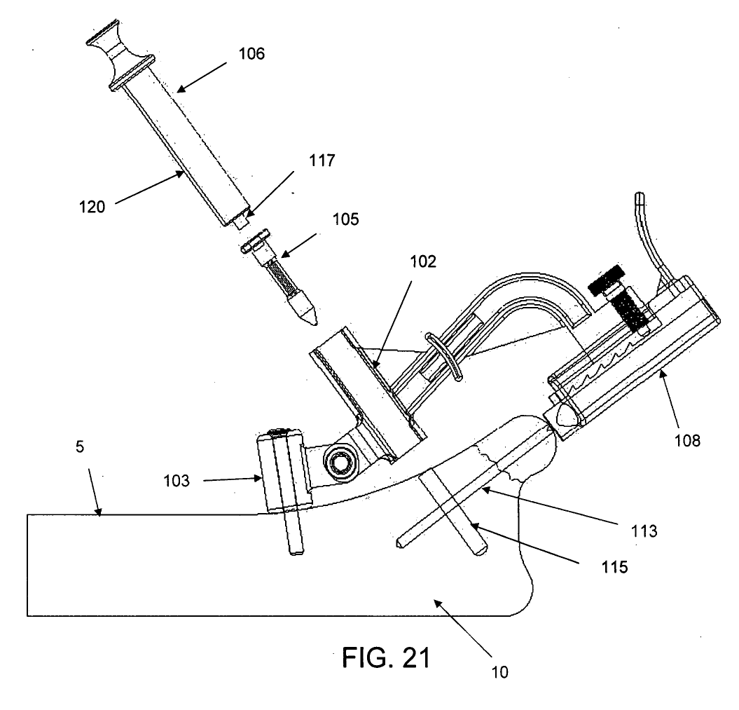

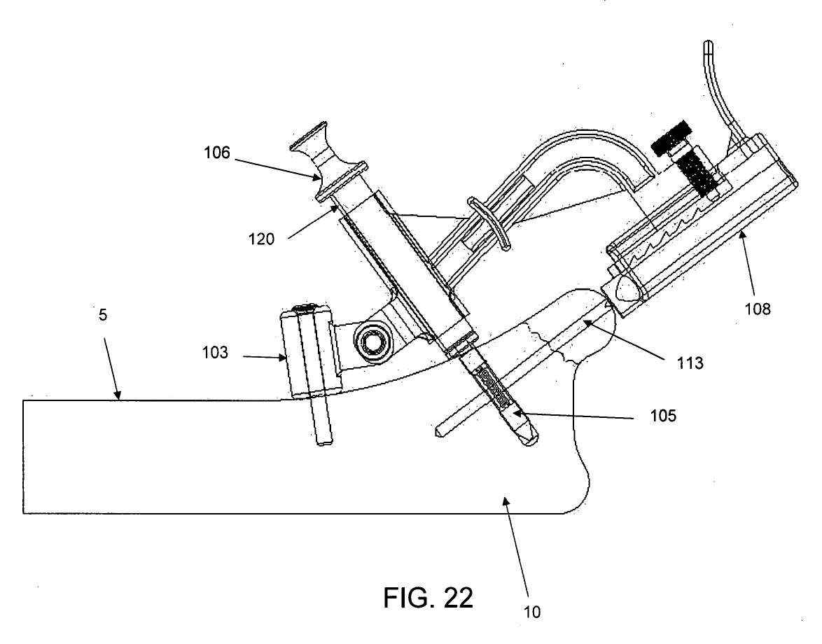

Next, and looking now at FIGS. 21 and 22, upper tube pusher 106 is used to advance a locking member 105 into one of the bores 115 formed in the tibia. As this occurs, locking member 105 is inserted so that its window 116 (FIG. 6) is aligned with one of the corresponding holes 113 in lateral malleolus 15 and distal end 10 of tibia 5. This is accomplished (i) by virtue of the alignment mechanism provided between upper tube pusher 106 and locking member 105 (i.e., a finger 117 provided on upper tube pusher 106 as shown in FIG. 21, and a socket 122 formed in locking member 105 as shown in FIG. 6) which aligns recess 120 (FIGS. 4 and 21) in upper tube pusher 106 and a recess 118 (FIG. 6) in locking member 105 when the two are temporarily co-joined, and (ii) by virtue of the alignment mechanism provided between arm 102 and upper tube pusher 106 (i.e., a rib 119 extending into the bore of arm 102 as shown in FIG. 4 and a recess 120 formed in upper tube pusher 106 as also shown in FIG. 4 and FIG. 21). Then a second locking member 105 is advanced in the same manner into the other of the bores 115, i.e., so that its window 116 is aligned with the corresponding hole 113 in lateral malleolus 15 and distal end 10 of tibia 5.

It should be appreciated that bores 115 and locking members 105 are preferably sized relative to one another so that locking members 105 make a secure friction fit in bores 115.

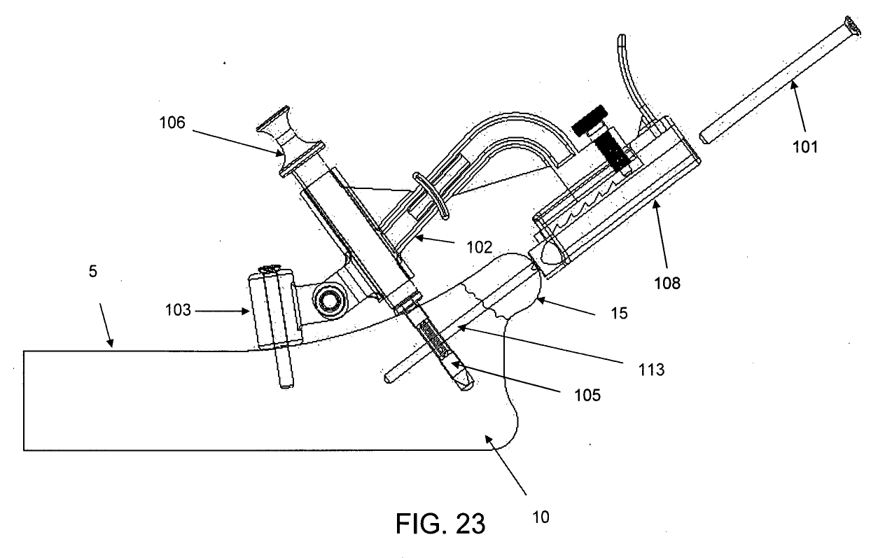

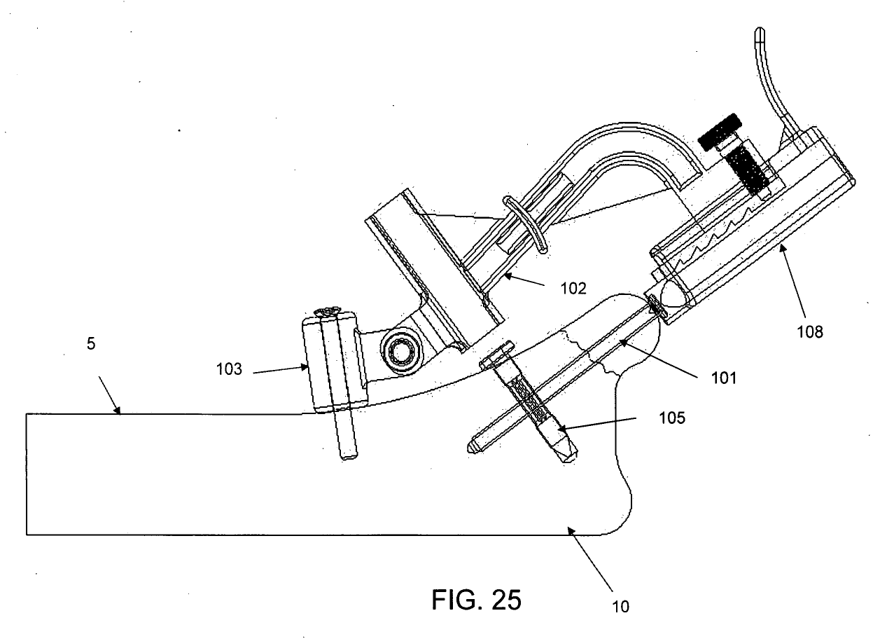

Looking next at FIGS. 23-25, an interlocking screw 101 is advanced through one of the bores in lower ratchet tubes 108 and through the aligned window 116 of a locking member 105. In this respect it will be appreciated that as interlocking screw 101 is advanced through window 116 of locking member 105, the threads of interlocking screw 101 engage projections 121 (FIG. 6) of locking member 105 so that interlocking screw 101 is secured to locking member 105. In this respect it will be appreciated that the distance between projections 121 (FIG. 6) is matched to the pitch of the thread on interlocking screw 101, whereby to allow the interlocking screw 101 to begin to thread into the gap between projections 121. Since the thread on the interlocking screw 101 is helical in nature, and the projections are in parallel planes, the interlocking screw 101 feeds between the projections 121 and binds with a slight interference fit, thereby locking screw 101 to locking member 105. Upper tube pusher 106 may then be withdrawn from arm 102 (FIG. 25). Then a second interlocking screw 101 is set through the other locking member in a similar manner.

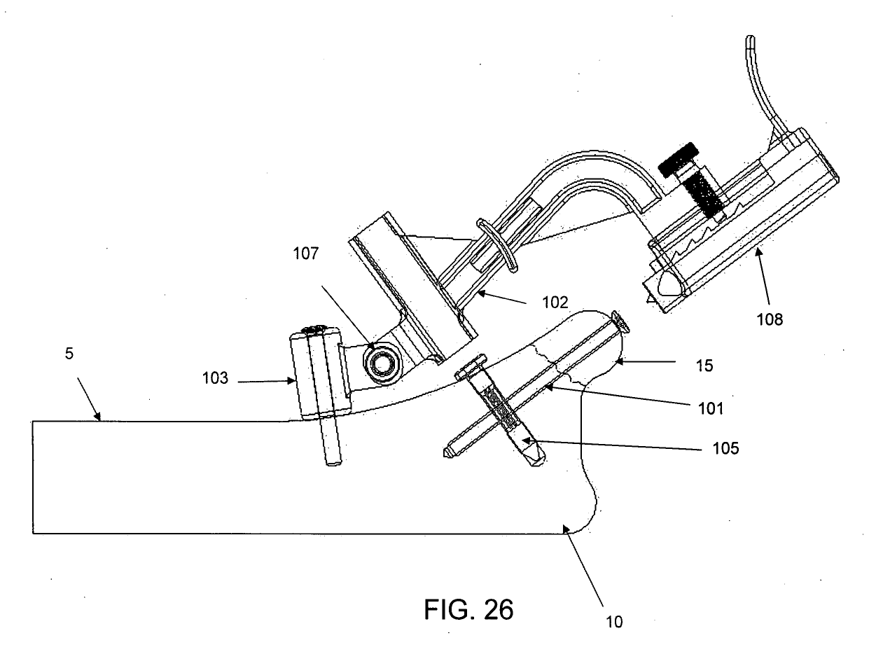

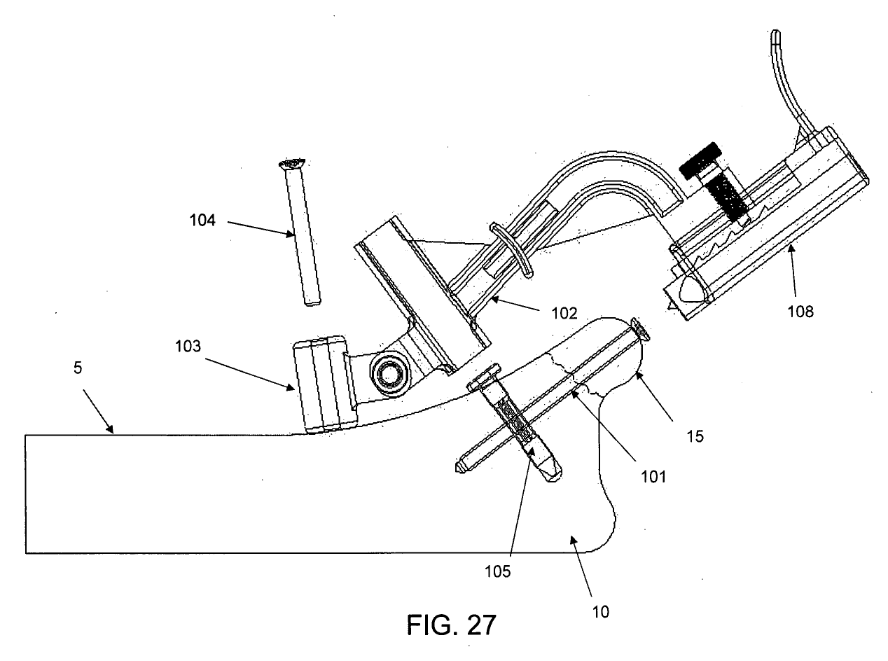

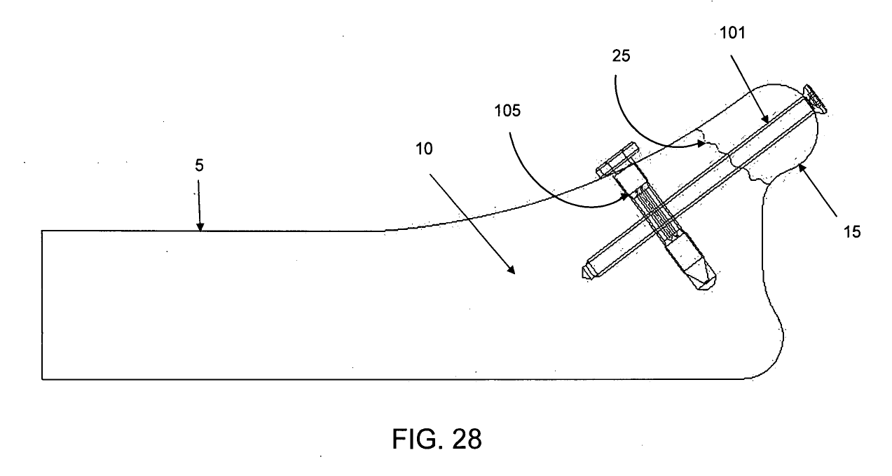

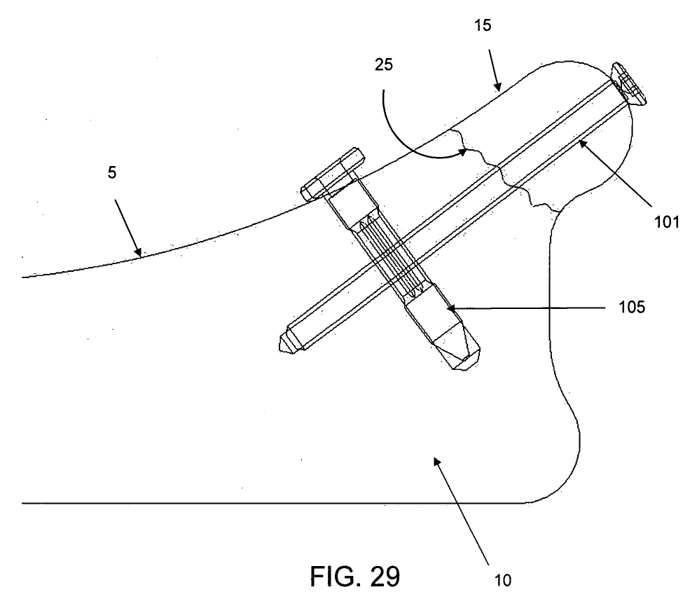

Next, and looking now at FIGS. 26-29, fracture fixation system 100 is manipulated so that lower ratchet tubes 108 are withdrawn from lateral malleolus 15 (FIG. 26), and then anchoring screw 104 is withdrawn (FIG. 27), and then the assembled arm 102/pivot block 103/lower ratchet tubes 108 are withdrawn (FIG. 28), thus leaving a pair of interlocking screws 101 extending through lateral malleolus 15, across fracture line 25, through a corresponding locking member 105 and into distal end 10 of tibia 5 (FIGS. 28 and 29), whereby to securely fix lateral malleolus 15 to the remainder of tibia 5 while healing occurs.

Significantly, since interlocking screws 101 are secured to locking members 105, effective fracture fixation can be achieved even where tibia 5 may have poor bone quality.

In the foregoing description, fracture fixation system 100 is constructed and used so as to set a pair of interlocking screws 101 into a pair of corresponding locking members 105. However, it will be appreciated that fracture fixation system 100 may be configured, and/or used, so as to set more or less interlocking screws 101 into corresponding locking members 105. Thus, for example, fracture fixation system 100 may be configured and/or used so as to set just one interlocking screw 101 into a corresponding locking member 105, or to set three interlocking screws 101 into corresponding locking members 105, etc.

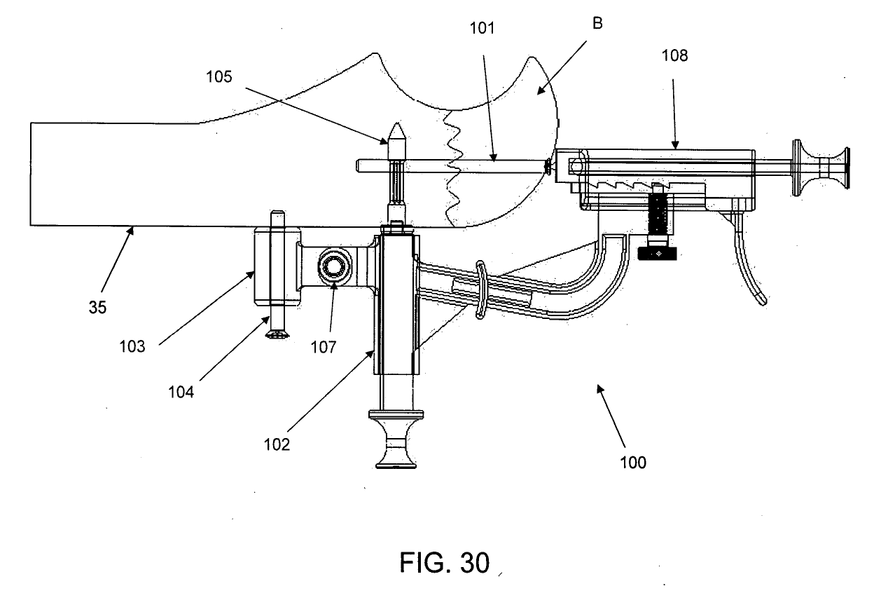

It should be appreciated that the present invention may be used for fracture fixations with bones other than the tibia. By way of example, but not limitation, and looking now at FIG. 30, fracture fixation system 100 may be used for fixation of a fracture of the olecranon B of the ulna 35. In essence, fracture fixation system 100 may be used with substantially any other bone or bones within the body where fracture fixation, and/or other bone fixation, is desired.

In this respect it should also be appreciated that the various system components are intended to be appropriately sized for the particular anatomical location within which the system is intended to be used.

Furthermore, it should also be appreciated that the various system components can have different form factors (i.e., shapes) appropriate for the particular anatomical limitations within which the system is intended to be used.

Additionally, the proximal ends of interlocking screws 101 and/or locking members 105 can be shaped so as to match the surface contours of the bone segments at the locations where they will be used. And in this respect it should also be appreciated that the bores 113 for seating interlocking screws 101, and/or the bores 115 for seating locking members 105, can be drilled and/or otherwise formed and/or finished (i.e., shaped) so as to permit the proximal heads of interlocking screws 101 and/or locking members 105 to be low profile, flush or below the surface of the adjacent bone.

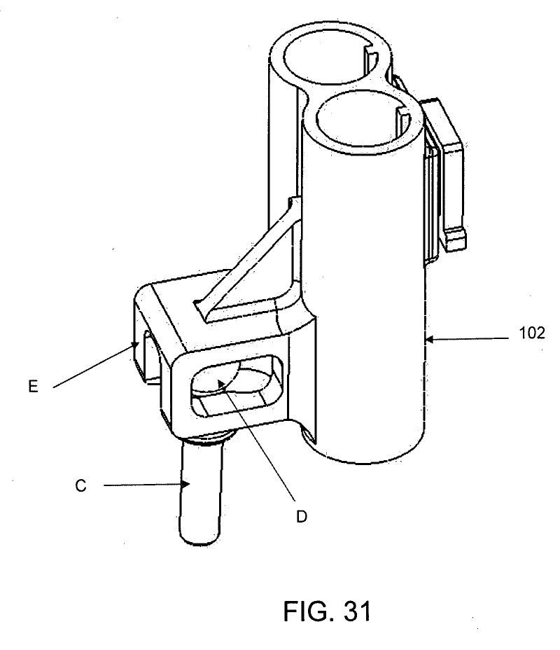

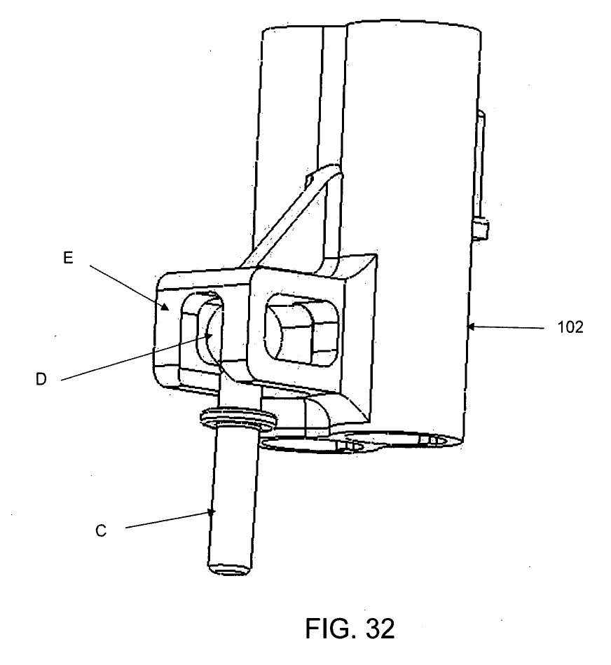

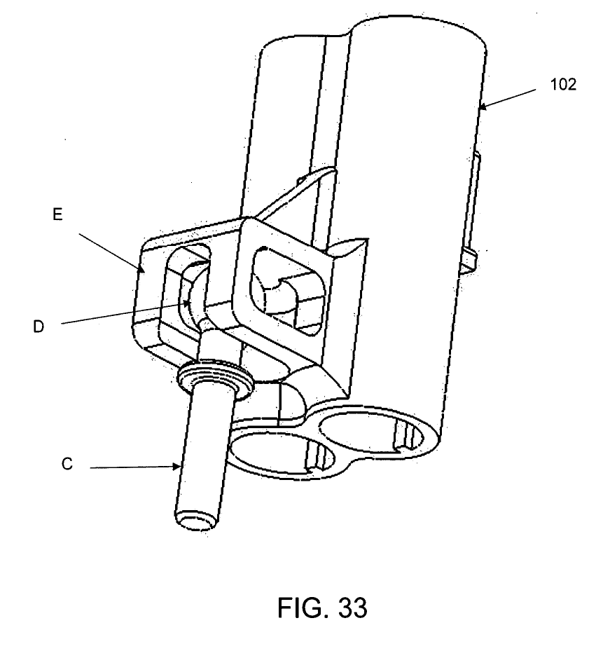

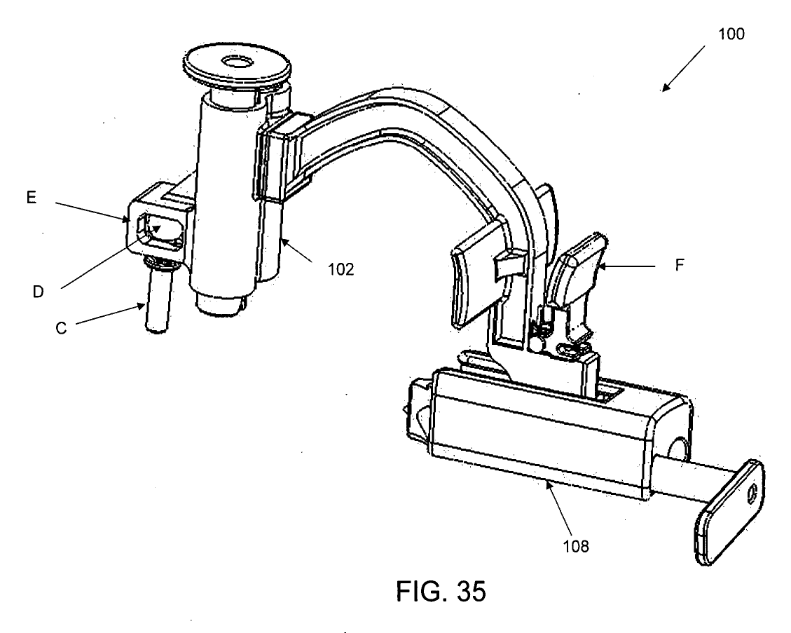

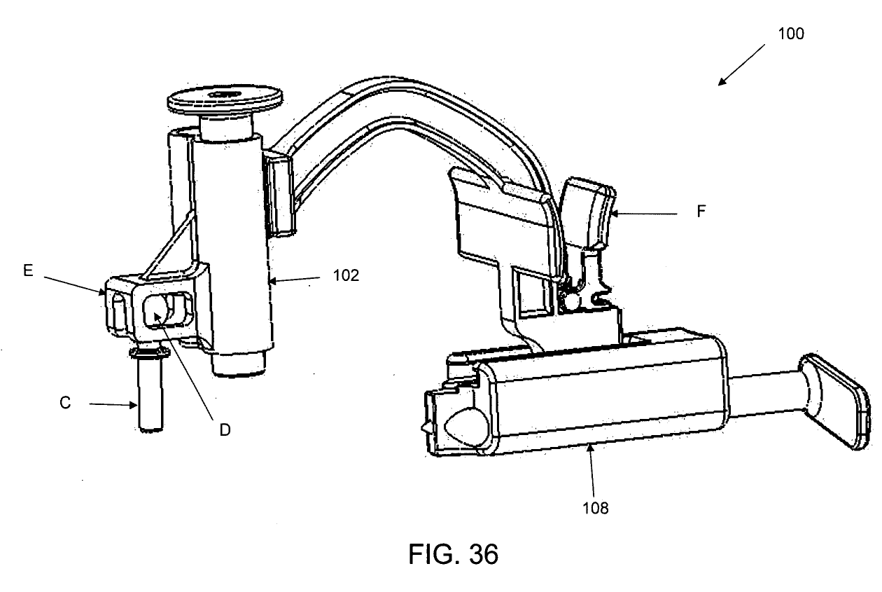

Looking next at FIGS. 31-36, it is also anticipated that arm 102 may be pivotally mounted to the host bone using an arrangement other than the aforementioned pivot block 103, anchoring pin 104, and clevis pin 107. Thus, for example, and looking now at FIGS. 31-36, pivot block 103 may be eliminated, anchoring pin 104 may be replaced by an anchoring pin C having a ball D formed thereon, and arm 102 may comprise a ball mount E configured to slip over, and rotatably pivot on, ball D. This arrangement can be advantageous, since it can permit arm 102 to be mounted closer to the host bone, as well as providing additional degrees of pivoting freedom.

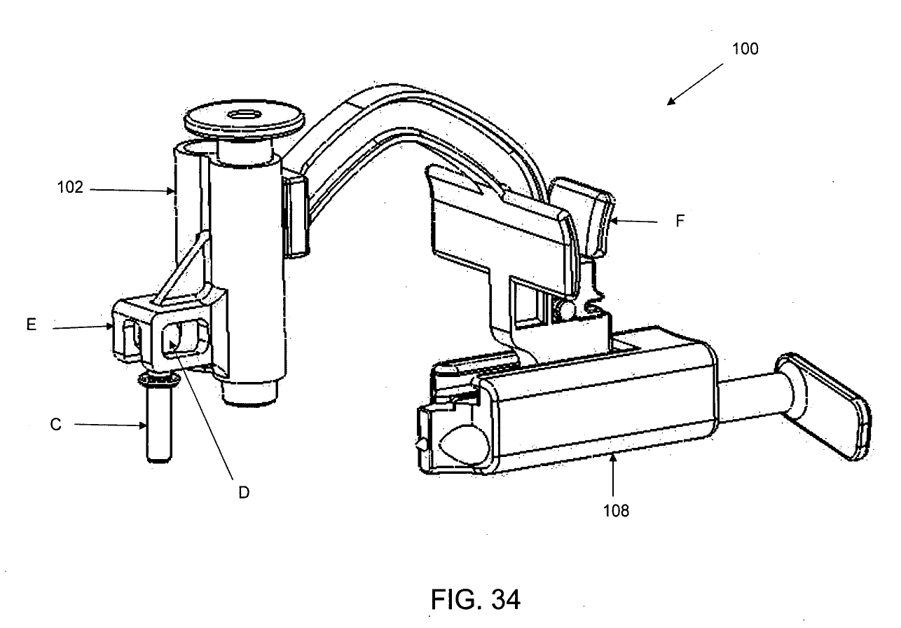

In addition to the foregoing, and looking now at FIGS. 34-36, it is also possible to selectively lock lower ratchet tubes 108 to arm 102 using other constructions. Thus, for example, a spring lever F may be used, where pressing spring lever F toward arm 102 enables movement of lower ratchet tubes 108 relative to arm 102. Correspondingly, releasing spring lever F causes lower ratchet tubes 108 to be locked in position relative to arm 102.

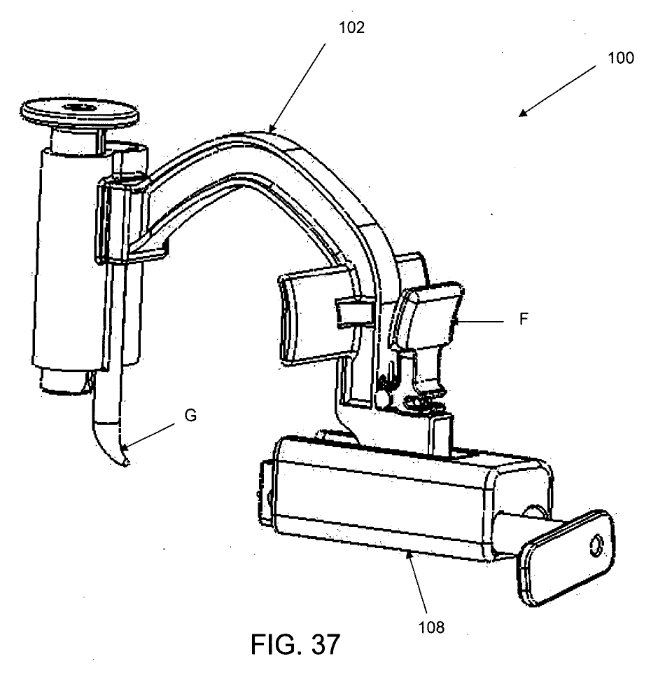

In yet another form of the invention, and looking now at FIG. 37, arm 102 may be pivotally mounted to the host bone using a finger G. In this form of the invention, a hole is made in the host bone, finger G is inserted into the hole, and then spring lever F (or other equivalent mechanism) is used to move lower ratchet tubes 108 against the fractured bone portion, whereby to hold the fractured bone portion securely against the host bone while it is secured using one or more locking members 105 and interlocking screws 101.

It should be appreciated that the various components of fracture fixation system 100 are formed out of materials which are consistent with the present invention. Thus, all of the components of fracture fixation system 100 (including locking members 105 and interlocking screws 101) may be formed out of metal. Alternatively, one or both of locking members 105 and/or interlocking screws 101 may be formed out of an absorbable material.

### MODIFICATIONS

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. A method for securing a first bone segment to a second bone segment, the method comprising the steps of:
positioning the first bone segment against the second bone segment, forming a first bore through the first bone segment and into the second bone segment, and forming a second bore into the second bone segment so that the second bore intersects the first bore;
positioning a locking member within the second bore so that an opening in the locking member is aligned with the first bore; and
positioning an interlocking screw within the first bore so that the interlocking screw passes through the opening in the locking member and makes a locking engagement therewith, whereby to lock the interlocking screw with the locking member and thereby secure the first bone segment to the second bone segment.

2. A method according to claim 1 wherein the locking member comprises at least one projection protruding into the opening formed in the locking member.

3. A method according to claim 2 wherein the at least one projection extends in a direction which is transverse to the longitudinal axis of the first bore.

4. A method according to claim 3 wherein the at least one projection also extends in a direction which is parallel to the longitudinal axis of the second bore.

5. A method according to claim 1 wherein the locking member comprises a pair of projections protruding into the opening formed in the locking member, and further wherein the pair of projections extend into the opening from opposing sides of the opening.

6. A method according to claim 5 wherein the pair of projections extend in directions which are transverse to the longitudinal axis of the first bore.

7. A method according to claim 6 wherein the pair of projections also extend in a direction which is parallel to the longitudinal axis of the second bore.

8. A method according to claim 5 wherein the pair of projections are separated by a distance which is matched to the pitch of the thread of the interlocking screw.

9. A method according to claim 1 wherein the first bone segment is held against the second bone segment when forming the first bore through the first bone segment and into the second bone segment and forming the second bore into the second bone segment.

10. A method according to claim 9 wherein a frame is used to hold the first bone segment against the second bone segment.

11. A method according to claim 10 wherein the frame comprises a first frame portion, a second frame portion and a third frame portion interconnected to one another, and further wherein the first frame portion is adapted to engage the second bone segment and the third frame portion is adapted to engage the first bone segment.

12. A method according to claim 10 wherein the second frame portion is pivotally connected to the first frame portion, and further wherein the third frame portion is slidably connected to the second frame portion.

13. A method according to claim 10 wherein the second frame portion comprises a drill guide for forming the second bore, and further wherein the third frame portion comprises a drill guide for forming the first bore.

14. A method according to claim 10 wherein the first frame portion is connected to the first bone segment with a pin.

15. A method according to claim 14 wherein the pin comprises a screw.

16. A method according to claim 15 wherein the screw comprises a ball head.

17. A method according to claim 14 wherein the pin comprises a finger for seating in a hole formed in the second bone segment.

18. A system for securing a first bone segment to a second bone segment, wherein a first bore is formed through the first bone segment and into the second bone segment, and a second bore is formed into the second bone segment so that the second bore intersects the first bore of the first bone segment, the system comprising:
a locking member for positioning within the second bore so that an opening in the locking member is aligned with the first bore; and
an interlocking screw for positioning within the first bore so that the interlocking screw may be passed through the opening in the locking member and make a locking engagement therewith, whereby to lock the interlocking screw with the locking member and thereby secure the first bone segment to the second bone segment.

19. A system according to claim 18 wherein the locking member comprises at least one projection protruding into the opening formed in the locking member.

20. A system according to claim 19 wherein the at least one projection extends in a direction which is transverse to the longitudinal axis of the first bore.

21. A system according to claim 20 wherein the at least one projection also extends in a direction which is parallel to the longitudinal axis of the second bore.

22. A system according to claim 18 wherein the locking member comprises a pair of projections protruding into the opening formed in the locking member, and further wherein the pair of projections extend into the opening from opposing sides of the opening.

23. A system according to claim 22 wherein the pair of projections extend in directions which are transverse to the longitudinal axis of the first bore.

24. A system according to claim 23 wherein the pair of projections also extend in a direction which is parallel to the longitudinal axis of the second bore.

25. A system according to claim 22 wherein the pair of projections are separated by a distance which is matched to the pitch of the thread of the interlocking screw.

26. A system according to claim 18 further comprising a frame for use in holding the first bone segment against the second bone segment.

27. A system according to claim 26 wherein the frame is configured to hold the first bone segment against the second bone segment when forming the first bore through the first bone segment and into the second bone segment, and forming the second bore into the second bone segment.

28. A system according to claim 26 wherein the frame comprises a first frame portion, a second frame portion and a third frame portion interconnected to one another, and further wherein the first frame portion is adapted to engage the second bone segment and the third frame portion is adapted to engage the first bone segment.

29. A system according to claim 28 wherein the second frame portion is pivotally connected to the first frame portion, and further wherein the third frame portion is slidably connected to the second frame portion.

30. A system according to claim 29 wherein the second frame portion comprises a drill guide for forming the second bore, and further wherein the third frame portion comprises a drill guide for forming the first bore.

31. A system according to claim 28 wherein the first frame portion is connected to the first bone segment with a pin.

32. A system according to claim 31 wherein the pin comprises a screw.

33. A system according to claim 32 wherein the screw comprises a ball head.

34. A system according to claim 31 wherein the pin comprises a finger for seating in a bone hole.
